(19) United States
(12) Patent Application Publication
(10) Pub. No.: US 2019/0117385 A1
(43) Pub. Date: Apr. 25, 2019

(54) VALVE DELIVERY SHEATH, SYSTEM, AND METHOD

(71) Applicant: Symetis SA, Ecublens (CH)

(72) Inventors: David Atlani, Jounge (FR); Pierre Simonin, Montflovin (FR); Jean-Luc Hefti, Cheseaux-Noreaz (CH); Stephane Delaloye, Bulach (CH); Luc Mantanus, Lausanne (CH); Stephane de Boer, Lausanne (CH)

(73) Assignee: Symetis SA, Ecublens (CH)

(21) Appl. No.: 16/170,473

(22) Filed: Oct. 25, 2018

Related U.S. Application Data
(60) Provisional application No. 62/576,874, filed on Oct. 25, 2017.

(51) Int. Cl. A61F 2/24

(52) U.S. Cl. CPC A61F 2/243; A61F 2230/0008; A61F 2002/9528; A61F 2230/0026; A61F 2230/0091; A61F 2230/0019

(57) ABSTRACT

The present disclosure pertains to delivery devices for prosthetic heart valves, components for such devices, and methods for manufacturing and delivering such components and devices.

### CROSS-REFERENCE TO RELATED APPLICATIONS

This application claims the benefit of priority under 35 U.S.C. § 119 to U.S. Provisional Application Ser. No. 62/576,874, filed Oct. 25, 2017, the entirety of which is incorporated herein by reference.

### TECHNICAL FIELD

The present disclosure pertains to delivery devices for prosthetic heart valves, components for such devices, and methods for manufacturing and delivering such components and devices.

### BACKGROUND

Catheter-deployed collapsible prosthetic heart valves are increasingly used in patients who may need a cardiac valve replacement, but who are not appropriate candidates for conventional open-chest, open-heart surgery. These collapsible and re-expandable prosthetic heart valves can be implanted transapically or percutaneously through the arterial system. Delivery through a patient's femoral artery is referred to as the transfemoral approach.

Proper positioning of the heart valve may be challenging to the interventional cardiologist. It would be desirable to be able to re-collapse a partially expanded heart valve in order to reduce the risk of trauma to the patient when repositioning or removing the heart valve.

Re-collapsing of a partially expanded heart valve may require relatively high axial pushing and pulling forces. This entails that the catheter tubing should be capable of safely and reliably transmitting these relatively high axial pushing and pulling forces from the handle or operator to the distal end of the heart valve delivery system.

These and other technical aspects are addressed in the present disclosure.

### SUMMARY OF THE DISCLOSURE

This summary is an overview of some of the teachings of the present disclosure and is not intended to be an exclusive or exhaustive treatment of the present subject matter. Further aspects, features and ideas are described in the detailed description and appended claims. Other aspects will be apparent to persons skilled in the art upon reading and understanding the following detailed description and viewing the drawings that form a part thereof, each of which is not to be taken in a limiting sense. Protection is claimed for any novel feature or idea described in the present disclosure and/or illustrated in the drawings, whether or not emphasis has been placed thereon.

One aspect of the disclosure describes a delivery system for delivering an expandable heart valve implant to a target site in a patient comprising: a flexible elongate tubular member having a proximal end and a distal end, an interior lumen extending between the proximal end and the distal end, and a polymeric material; wherein the elongate tubular member comprises a plurality of metal strands (e.g. wires) which are arranged in a side-by-side relationship and helically wound along the longitudinal axis of the elongate tubular member to define an outer surface having a plurality of helically oriented depressions; and wherein the polymeric material is disposed on the elongate tubular member such that at least part of the helically oriented depressions of the outer surface of the elongate tubular member are at least partially filled.

One aspect of the disclosure describes a delivery system for delivering an expandable heart valve implant to a target site in a patient comprising: a flexible elongate tubular member having a proximal end and a distal end, and an interior lumen extending between the proximal end and the distal end; wherein the elongate tubular member comprises a plurality of metal strands (e.g. wires) which are arranged in a side-by-side relationship and helically wound along the longitudinal axis of the elongate tubular member; and wherein a portion of the plurality of metal strands are joined to at least one circumferentially adjacent strand by welding, soldering, or brazing. In one embodiment, the flexible elongate tubular member further comprises a polymeric material which is disposed on the elongate tubular member. The strands may define an outer surface of the elongate tubular member to have a plurality of helically oriented depressions. At least part of the helically oriented depressions of the outer surface of the elongate tubular member may be at least partially filled by the polymeric material.

In some embodiments, the polymeric material may form a layer on at least a part of the outer surface of the elongate tubular member.

One aspect of the disclosure describes a delivery system for delivering an expandable heart valve implant to a target site in a patient comprising: a flexible elongate tubular member having a proximal end and a distal end, an interior lumen extending between the proximal end and the distal end; wherein the elongate tubular member comprises a plurality of metal strands (e.g. wires) which are arranged in a side-by-side relationship and helically wound along the longitudinal axis of the elongate tubular member, the metal strands configured to bear axial loads by edge-to-edge contact between adjacent strands; and a substantially inelastic polymeric sleeve disposed coaxially around the elongate tubular member along at least a part of the length of the tubular member and in intimate contact with the exterior surface of the metal strands, for reinforcing the metal strands to constrain the tubular member against radial expansion under axial compression load.

Resisting radial expansion may enhance the column strength of the elongate tubular member and/or stabilize the elongate tubular member against axial shortening.

As used herein, the term “inelastic” may refer to an elastic modulus of at least about 1 GPa. Optionally, the polymer material of the jacket may have an elastic modulus of at least about 1.5 GPa, optionally at least about 2 GPa, optionally at least about 2.5 GPa.

As already described, the strands may optionally define an outer surface of the elongate tubular member to have a plurality of helically oriented depressions. At least part of the helically oriented depressions of the outer surface of the elongate tubular member may be at least partially filled by the polymeric material.

Also as already described, a portion of the plurality of metal strands may optionally be joined to at least one circumferentially adjacent strand by welding, soldering, or brazing.

In some embodiments, at least one of the plurality of metal strands may have a non-circular cross-sectional shape. In some embodiments, the plurality of metal strands may comprise strands having a rectangular shape, a rectangular shape with rounded edges or rounded corners, an oval shape, or a trapezoidal shape (optionally with rounded edges or rounded corners).

In some embodiments, the elongate tubular member may comprise a single layer of helically wound metal strands. Alternatively, the elongate tubular member may comprise two layers of helically wound metal strands.

In some embodiments, the elongate tubular member may comprise a side-by-side arrangement of at least 2, in particular of 5 to about 20, strands.

In some embodiments, a portion of the plurality of metal strands may be joined to at least one circumferentially adjacent strand by welding, soldering, brazing, or by a substantially inelastic adhesive.

In some embodiments, portion of the plurality of metal strands may be joined to at least one circumferentially adjacent strand in a pattern of joints, wherein said pattern may extend substantially continuously or intermittently between at least a portion of the outer surface of the elongate tubular member. Additionally or alternatively, the pattern may be longitudinally oriented in a straight or non-straight line between the proximal end and the distal end of the elongate tubular member, circumferentially oriented in a straight or non-straight line, helically oriented anti-clock-wise with respect to the helically wound plurality of metal strands, or helically oriented clock-wise with respect to the helically wound plurality of metal strands but with a different pitch than the helically wound plurality of metal strands. Additionally or alternatively, the pattern may be such that at least a portion of the plurality of metal strands is welded at 1 to about 8 locations to a circumferentially adjacent strand per circumference of said strand.

In some embodiments, the elongate tubular structure may have an elongation along its longitudinal axis of less than about 0.5% when subjected to a tensile load of about 100 N. In some embodiments, the delivery system further comprises a mount attached to the elongate tubular member at or approximate to its distal end which is configured to transmit a tensile load of at least about 100 N to a further part of the delivery system which is attached to the mount.

In some embodiments, the delivery device further comprises second flexible elongate tubular member having a second proximal end and a second distal end and comprising a second interior lumen extending between the second proximal end and the second distal end, wherein the second elongate tubular member comprises a second plurality of metal strands which are arranged in a side-by-side relationship and helically wound along the longitudinal axis of the second elongate tubular member to define a second outer surface having a second plurality of helically oriented depressions; wherein the second elongate tubular member is arranged in the interior lumen of the first elongate tubular member or wherein the first elongate tubular member is arranged in the second interior lumen of the second elongate tubular member.

In some embodiments, the delivery system further comprises a housing for carrying the heart valve implant and a handle for operating the delivery system.

In some embodiments, the delivery system is configured to at least partially re-collapse the heart valve implant after its at least partial expansion.

One aspect of the disclosure describes a method of repositioning or withdrawing an at least partially expanded implant during the delivery of an expandable heart valve implant to a target site in a patient comprising applying a tensile force to the elongate tubular member of a delivery system as described above which causes the at least partially expanded heart valve implant to re-collapse at least partially, followed by repositioning or withdrawing of the implant.

One aspect of the disclosure describes a method for manufacturing a delivery system as described above, comprising providing a flexible elongate tubular member having a proximal end and a distal end and comprising an interior lumen extending between the proximal end and the distal end, wherein the elongate tubular member comprises a plurality of metal strands which are arranged in a side-by-side relationship and helically wound along the longitudinal axis of the elongate tubular member to define an outer surface having a plurality of helically oriented depressions; and: i) providing a polymeric material, and, in any order: heating the polymeric material to a flowable state, and applying the polymeric material onto at least a part of the outer surface of the elongate tubular member, such that at least part of the helically oriented depressions of the outer surface are at least partially filled; or ii) joining a portion of the plurality of metal strands to at least one circumferentially adjacent strand by welding, soldering, or brazing.

One aspect of the disclosure describes a catheter for transfemoral delivery of a heart valve implant comprising an elongate tubular member having a proximal end and a distal end; a housing component for carrying the heart valve implant which is attached to the distal end of the elongate tubular member; wherein the elongate tubular member comprises a first section at or approximate to its distal end and a second section proximally adjacent to the first section; wherein the first section has a length in longitudinal direction of between about 5 and about 50 mm, an internal diameter of between about 2 and about 6 mm, is more flexible than the second section, and is configured to bend without kinking to an angle of at least about 45° relative to its longitudinal axis.

One aspect of the disclosure describes a catheter for transfemoral delivery of a heart valve implant comprising an elongate tubular member having a proximal end and a distal end; a housing component for carrying the heart valve implant which is attached to the distal end of the elongate tubular member; wherein the elongate tubular member comprises a first section at or approximate to its distal end and a second section proximally adjacent to the first section; wherein the first section has a length in longitudinal direction of between about 5 and about 50 mm, an internal diameter of between about 2 and about 6 mm, and a reduced wall thickness in comparison to the second section.

One aspect of the disclosure describes a catheter system comprising the catheter as described above, a handle, and optionally an implantable heart valve.

One aspect of the disclosure describes a method of manufacturing a catheter or catheter system as described above, wherein a first and a second tubular member may be provided and wherein the first and the second tubular member may be attached to each other.

One aspect of the disclosure describes an interface member for a system for delivering a medical device to a target site in a patient, the interface member being biased towards a radially expanded condition for (i) bridging a gap between the holder and the sheath when in the second position, and/or (ii) defining a generally smooth interface surface between the holder and the sheath when in the second position.

The above summary of some embodiments and aspects is not intended to describe each disclosed embodiment or every implementation of the present disclosure. The drawings and the detailed description, which follow, more particularly exemplify these embodiments.

### BRIEF DESCRIPTION OF DRAWINGS

FIG. 1a shows an exemplary illustration of a system 100 for delivering an expandable heart valve implant to a target site in a patient.

FIG. 1b shows an exemplary 3-dimensional representation of a flexible elongate tubular member 120.

FIG. 2 shows an exemplary cross-sectional representation of a flexible elongate tubular member 120.

FIG. 3 shows an exemplary schematic illustration of a flexible elongate tubular member 120.

FIG. 4 shows an exemplary illustration of a flexible elongate tubular member 120 comprising a polymeric material 140 disposed on the elongate tubular member 120.

FIGS. 5a to 5d show exemplary schematic illustrations of patterns of joints of elongate tubular member 120.

FIG. 6 shows an exemplary illustration of a catheter 200 for transcatheter (e.g. transfemoral) delivery of a heart valve implant.

FIG. 7 shows an exemplary illustration of an interface member 300 in front view.

FIG. 8 shows an exemplary illustration of an interface member 300 in cross-sectional view along line A of FIG. 7.

### DETAILED DESCRIPTION

The following detailed description should be read with reference to the drawings, which are not necessarily to scale, wherein like reference numerals indicate like elements throughout the several views. The detailed description and drawings are intended to illustrate but not limit the claimed invention. Those skilled in the art will recognize that the various elements described and/or shown may be arranged in various combinations and configurations without departing from the scope of the disclosure. The detailed description and drawings illustrate example embodiments of the claimed invention.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (i.e., having the same function or result). In many instances, the term “about” may include numbers that are rounded to the nearest significant figure. Other uses of the term “about” (i.e., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g. 1 to 5 includes 1, 1.5, 2, 2.75, 3, 3.80, 4, and 5).

Although some suitable dimensions, ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural references unless the content clearly dictates otherwise. As used in this specification and the appended claims, the term “or” is generally employed in its sense including “and/or” unless the content clearly dictates otherwise.

Relative terms such as “proximal”, “distal”, “advance”, “withdraw”, “retract”, variants thereof, and the like, may generally be considered with respect to the positioning, direction, and/or operation of various elements relative to a user/operator/manipulator of the device, wherein “proximal”, “withdraw” and “retract” indicate or refer to closer to or toward the user and “distal” and “advance” indicate or refer to farther from or away from the user. Other relative terms, such as “upstream” and “downstream” refer to a direction of fluid flow within a lumen, such as a body lumen or blood vessel.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect such feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

One aspect of the disclosure pertains to a delivery system for delivering an expandable heart valve implant to a target site in a patient comprising a flexible elongate tubular member which is capable of safely and reliably transmitting relatively high axial pushing and pulling forces from the handle or operator to the distal end of the delivery system.

In some embodiments, the delivery system for delivering an expandable heart valve implant to a target site in a patient may comprise a flexible elongate tubular member having a proximal end and a distal end, and an interior lumen extending between the proximal end and the distal end. The elongate tubular member may comprise a plurality of metal strands (e.g. wires) which are arranged in a side-by-side relationship and helically wound along the longitudinal axis of the elongate tubular member.

In some embodiments, an (optionally substantially inelastic) polymeric sleeve may be disposed coaxially around the elongate tubular member, along at least a part of the length of the tubular member and in intimate contact with the exterior surface of the metal strands.

Optionally, the metal strands may be configured to bear axial loads by edge-to-edge contact between adjacent strands. A substantially inelastic polymeric sleeve may reinforce the metal strands to resist radial expansion of the tubular member under axial compression load. Resisting radial expansion may enhance the column strength of the elongate tubular member and/or stabilize the elongate tubular member against axial shortening.

Whether or not the polymeric sleeve is substantially inelastic (but advantageously in combination), the strands may optionally define an outer surface of the elongate tubular member having a plurality of helically oriented depressions. The polymeric sleeve may be disposed on the elongate tubular member such that at least part of the helically oriented depressions are at least partially filled. Such intermeshing between the sleeve and the depressions can enhance the interference fit between the sleeve and the elongate tubular member, and resist axial and/or torsional displacement of the strands relative to the sleeve, thereby to increase the strength for bearing axial compression and/or elongation loads. The interference fit between the polymeric sleeve and the elongate tubular member may be optionally obtained by heat-shrinking a heat-shrinkable polymeric sleeve onto the elongate tubular member.

Whether or not a polymeric sleeve is provided (but advantageously in combination), a portion of the plurality of metal strands may be joined to at least one circumferentially adjacent strand by welding, soldering, or brazing. Such joining may provide reduce sliding between adjacent strands, at localized positions, to resist thinning and/or elongation and/or torsional sliding of the strands under axial load, while still maintaining flexibility.

Additionally or alternatively, in some embodiments, the delivery system for delivering an expandable heart valve implant to a target site in a patient may comprise a flexible elongate tubular member having a proximal end and a distal end, an interior lumen extending between the proximal end and the distal end, and a polymeric material. The elongate tubular member may comprise a plurality of metal strands (e.g. wires) which are arranged in a side-by-side relationship and are helically wound along the longitudinal axis of the elongate tubular member to define an outer surface having a plurality of helically oriented depressions. The polymeric material may be disposed on the elongate tubular member such that at least part of the helically oriented depressions of the outer surface of the elongate tubular member are at least partially filled. The polymeric material may polymeric sleeve.

Additionally or alternatively, in some embodiments, the delivery system for delivering an expandable heart valve implant to a target site in a patient may comprise a flexible elongate tubular member having a proximal end and a distal end, and an interior lumen extending between the proximal end and the distal end. The elongate tubular member may comprise a plurality of metal strands (e.g. wires) which are arranged in a side-by-side relationship and are helically wound along the longitudinal axis of the elongate tubular member to define an outer surface having a plurality of helically oriented depressions. A portion of the plurality of metal strands may be joined to at least one circumferentially adjacent strand by welding, soldering, or brazing. In one embodiment, the flexible elongate tubular member may additionally comprise a polymeric material which may be disposed on the elongate tubular member such that at least part of the helically oriented depressions of the outer surface of the elongate tubular member are at least partially filled.

Additionally or alternatively, in some embodiments, the delivery system for delivering an expandable heart valve implant to a target site in a patient may comprise a flexible elongate tubular member having a proximal end and a distal end, an interior lumen extending between the proximal end and the distal end; wherein the elongate tubular member comprises a plurality of metal strands (e.g. wires) which are arranged in a side-by-side relationship and helically wound along the longitudinal axis of the elongate tubular member, the metal strands configured to bear axial loads by edge-to-edge contact between adjacent strands; and a substantially inelastic polymeric sleeve disposed coaxially around the elongate tubular member, in intimate contact with the exterior surface of the metal strands, for reinforcing the metal strands to constrain the tubular member against radial expansion under axial compression load. Resisting radial expansion may enhance the column strength of the elongate tubular member and/or stabilize the elongate tubular member against axial shortening.

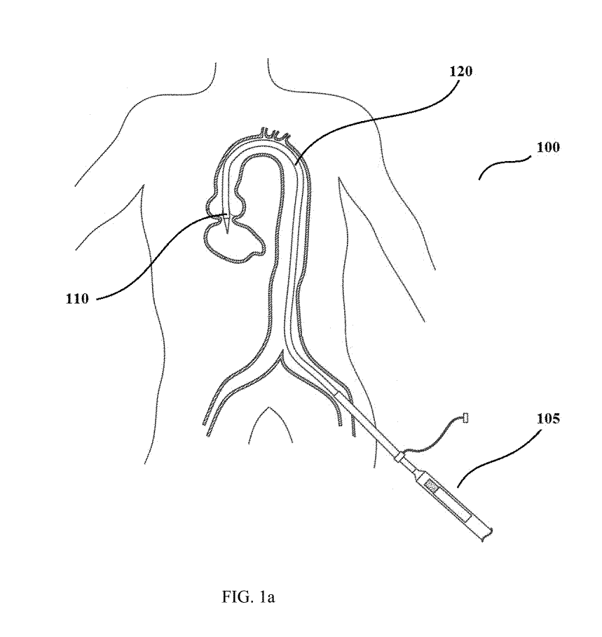

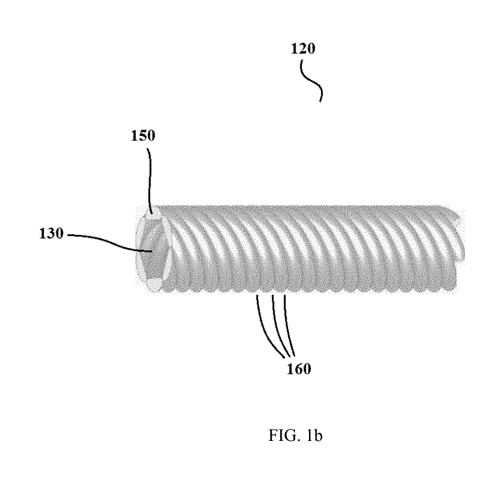

FIG. 1a shows an exemplary illustration of a delivery system 100 for delivering an expandable heart valve implant to a target site in a patient. The delivery system 100 is shown inserted into the patient with a housing (e.g. a sheath) for carrying the heart valve implant 110 being positioned in the vicinity of the aortic valve. In the exemplary illustration of FIG. 1a, system 100 further comprises a handle 105 for operating the delivery system 100 and an elongate tubular member 120 (e.g. a catheter tubing) connecting the handle 105 to the housing 110.

FIG. 1b shows a non-limiting illustration of one exemplary type of a flexible elongate tubular member 120 that may be used in a delivery system 100. The flexible elongate tubular member 120 has a proximal end and a distal end and an interior lumen 130 extending between the proximal end and the distal end. The tubular member 120 comprises a plurality of metal strands 150 which are arranged in a side-by-side relationship and are helically wound along the longitudinal axis of the elongate tubular member 120 to define an outer surface having a plurality of helically oriented depressions 160. FIG. 1 shows a side-by-side relationship of six strands 150, but embodiments are not so limited. The flexible elongate tubular member 120 can alternatively include 2, 3, 4, 5, 7, 8, 9, 10, 12, 15 or more strands 150. In FIG. 1, the plurality of strands 150 is arranged in a side-by-side relationship such that the strands 150 are in contact to each other. The contact may be edge-to-edge, as shown in FIG. 1b. In FIG. 1b, the plurality of strands 150 is arranged in a side-by-side relationship such that the strands 150 form a single layer, but embodiments are not so limited. The flexible elongate tubular member 120 can alternatively include two or more layers of strands 150 wherein the strands in each layer are helically wound along the longitudinal axis of the elongate tubular member 120 and arranged in a side-by-side relationship. In an example, the cross-sectional shape of the strands 150 may be oval or ovalized. The plurality of strands 150 are arranged in a side-by-side relationship such that cross-sectional shapes of the plurality of strands 150 define a full circumference around the longitudinal axis of the elongate tubular member 120 without multiple participation of any of said plurality of strands.

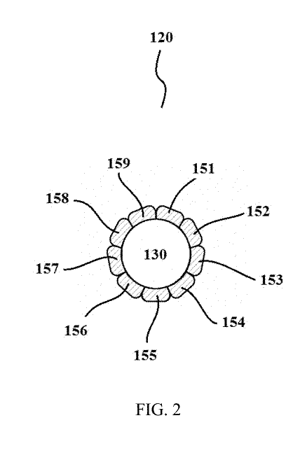

The latter feature, i.e. a plurality of strands 150 which are arranged in a side-by-side relationship such that cross-sectional shapes of the plurality of strands 150 define a full circumference around the longitudinal axis of the elongate tubular member 120 multiple participation of any of said plurality of strands, is also exemplarily illustrated in FIG. 2. FIG. 2 shows a cross-sectional view of a flexible elongate tubular member 120 comprising a plurality of nine metal strands 151 to 159 which are arranged in a side-by-side relationship around interior lumen 130. Strands 151 to 159 are helically wound along the longitudinal axis of the elongate tubular member 120 to define an outer surface having a plurality of helically oriented depressions. The strands 151 to 159 are arranged in a side-by-side relationship such that cross-sectional shapes of the plurality of strands define a full circumference without multiple participation of any of strands 151 to 159 to define said circumference. That is to say that each of strands 151 to 159 is present no more than once in a cross-sectional representation (orthogonal to the longitudinal axis) of the elongate tubular member 120.

In some embodiments, an elongate tubular member comprising a plurality of metal strands which are arranged in a side-by-side relationship and are helically wound along the longitudinal axis of the elongate tubular member (and optionally to define an outer surface having a plurality of helically oriented depressions) may have sufficient flexibility to be suitable as catheter tubing material. In an example, disposing a polymeric material or a polymeric sleeve on the elongate tubular member such that at least part of the helically oriented depressions of the outer surface of the elongate tubular member are at least partially filled may prohibit or prevent the metal strands from sliding against each other, such as under load. In another example, joining a portion of the plurality of metal strands to at least one circumferentially adjacent strand by welding, soldering, or brazing may aid in prohibiting or preventing the metal strands from sliding against each other. Both measures may reduce the degree of mobility of the individual strands and, thus, may limit dimensional changes under compression load and extension load. Disposing a polymeric material on the elongate tubular member such that at least part of the helically oriented depressions of the outer surface of the elongate tubular member are at least partially filled may be particularly suited to reduce bulging and shortening under axial compression load. Joining a portion of the plurality of metal strands to at least one circumferentially adjacent strand by welding, soldering, or brazing may be particularly suited to reduce narrowing down and elongation under axial extension load.

In some embodiments, the elongate tubular member may comprise a side-by-side relationship of at least 2, in particular of about 5 to about 25, or about 6 to about 22 or about 7 to about 20, strands. The plurality of strands may be arranged in a side-by-side relationship such that the cross-sectional shapes of the plurality of strands define a full circumference around the longitudinal axis of the elongate tubular member without multiple participation of any of said plurality of strands. Such a side-by-side arrangement defining a full circumference around the longitudinal axis of the elongate tubular member may consist of at least 2, in particular of about 5 to about 25, or about 6 to about 22 or about 7 to about 20, strands.

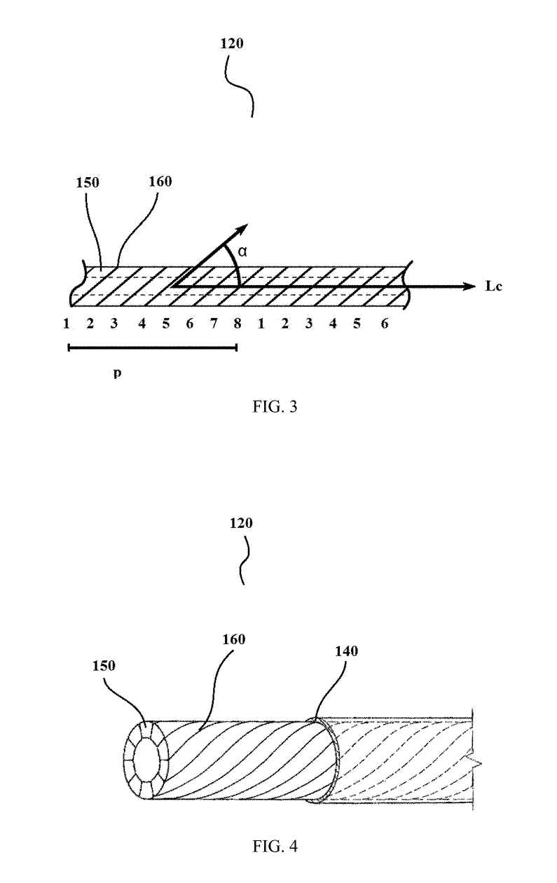

FIG. 3 is an exemplary representation of an elongate tubular member 120 which is composed of a single layer of a plurality of eight strands 150 which is arranged in a side-by-side relationship.

The individual strands are labelled “1” to “8”. As can be seen in FIG. 3, the side-by-side-arrangement of strands 1 to 8 repeats itself within axial distance p. This means that it takes distance p for an individual strand to describe a full circumference of elongate tubular member 120. Distance p may also be called helical pitch and is the axial distance of one complete helical turn of an individual strand. FIG. 3 further shows a plurality of metal strands 150 which are arranged to define an outer surface having a plurality of helically oriented depressions 160. The depressions are oriented at an angle α with respect to the longitudinal axis L_{c} of elongate tubular member 120. In some embodiments, p may be within the range of about 3 mm to about 100 mm, in particular 5 mm to 80 mm, or about 6 mm to about 60 mm. In some embodiments, α may be between about 5° and about 85°, in particular about 15° to about 75°, or about 25° to about 75°, or about 35° to about 65°.

In some embodiments, the elongate tubular member may have a single layer of helically wound metal strands.

In some embodiments, the elongate tubular member may have two layers of helically wound metal strands. In this case, the elongate tubular member may comprise an inner layer of helically wound strands having a first side-by-side arrangement defining a first full circumference around the longitudinal axis of the elongate tubular member and a second outer layer of helically wound strands having a second side-by-side arrangement defining a second full circumference around the longitudinal axis of the elongate tubular member. The first and second side-by-side arrangements defining a full circumference around the longitudinal axis of the elongate tubular member may independently from each other consist of at least 2, in particular of 5 to about 20, or about 6 to about 18 or about 7 to about 16, strands. The orientation of the helical windings of the first and second side-by-side arrangements to each other may be anti-clock-wise or clock-wise with the same or a different pitch. The helical windings may be wound in the same sense, or in opposite senses.

In some embodiments, the elongate tubular member may comprise a plurality of strands comprising a biocompatible metal. Exemplary metals include stainless steel, e.g. 316L, Nitinol, titanium, and other metals and alloys. Such elongate tubular members are commercially available, for instance from Fort Wayne Metals, Indiana, in the United States.

In some embodiments, at least a portion of the plurality of metal strands may have a non-circular cross-sectional shape, in particular a rectangular shape, a rectangular shape with rounded edges, an oval shape, a trapezoidal shape (optionally with rounded edges or rounded corners). A non-circular cross-sectional shape may increase the frictional forces between the strands of the plurality of strands and, thus, may reduce the degree of mobility of the individual strands. In some embodiments, the plurality of strands may be arranged in a side-by-side relationship such that the strands are in contact to each other. In some embodiments, the plurality of strands may be arranged in a side-by-side relationship such that the strands are in contact to each other along a full circumference of the strands. In some embodiments, the plurality of strands may be arranged in a side-by-side relationship such that at least part of the strands are in proximity to each other wherein the space between said strands is filled with a second polymeric material to reduce the degree of mobility of said strands. The second polymeric material may be same material as or different to the polymeric material that may be disposed on the elongate tubular member such that at least part of the helically oriented depressions of the outer surface of the elongate tubular member are at least partially filled. The second polymeric material may be same material as or different to the polymeric sleeve.

In some embodiments, the arrangement of plurality of strands of the elongate tubular member may be substantially circular or circular. In some embodiments, the arrangement of plurality of strands of the elongate tubular member may have an outer diameter of about 0.8 mm to about 10 mm, in particular about 0.9 mm to about 8 mm, or about 1.0 mm to about 6 mm, or about 1.1 mm to about 4 mm, or about 1.2 mm to about 2 mm. In some embodiments, the arrangement of plurality of strands of the elongate tubular member may have an inner diameter of about 0.6 mm to about 8 mm, in particular about 0.7 mm to about 6 mm, or about 0.8 mm to about 4 mm, or about 0.9 mm to about 2 mm.

In some embodiments, the plurality of strands may comprise strands having a diameter (or in case of rectangular or trapezoidal shapes: diagonal) of about 0.05 mm to 0.5 mm, in particular about 0.08 mm to about 0.4 mm, or about 0.1 mm to about 0.3 mm, or about 0.12 mm to about 0.25 mm.

In some embodiments, the elongate tubular member may have a length of between about 70 cm and 150 cm, in particular between about 80 cm and 140 cm, or between about 90 cm and 130 cm, or between about 100 cm and 120 cm.

In some embodiments, the elongate tubular member may comprise a single layer of a plurality of at least 2, in particular about 6 to about 22, metal strands, wherein the plurality of strands each have a non-circular cross-sectional shape, in particular a rectangular shape, a rectangular shape with rounded edges, an oval shape, or a trapezoidal shape (optionally with rounded edges or rounded corners) and wherein the plurality of strands may be arranged in a side-by-side relationship such that the strands are in contact to each other.

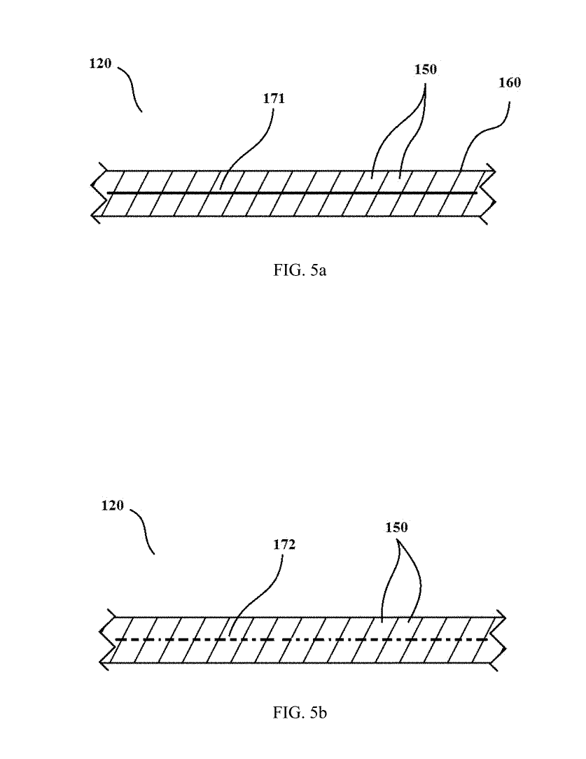

FIG. 4 shows a non-limiting illustration of one exemplary type of a flexible elongate tubular member 120 wherein the elongate tubular member 120 comprises a plurality of metal strands 150 which are arranged in a side-by-side relationship and helically wound along the longitudinal axis of the elongate tubular member 120. As schematically shown in FIG. 4, the side-by-side arrangement of strands 150 defines an outer surface having a plurality of helically oriented depressions 160. In FIG. 4, a polymeric material 140 is disposed on the elongate tubular member 120 such that at least part of the helically oriented depressions 160 of the outer surface of the elongate tubular member 120 are at least partially filled. In the exemplary embodiment shown in FIG. 4, the polymeric material 140 is arranged as a layer or sleeve filling at least part of the depressions 160 with polymeric material 140.

In some embodiments, the polymeric material may comprise a thermoplastic material or a thermoset material. The polymeric material may be filled or unfilled. Suitable fillers may be reinforcing fillers, fillers adjusting tensile properties, compressive strength, and flexural strength.

In some embodiments, the polymeric material may comprise a thermoplastic polymeric material, in particular a polyether ether ketone, a polyether, a polyester, a polyamide, a polyolefin, or a combination thereof. In some embodiments, the polymeric material may comprise a polyether ether ketone.

In some embodiments, the polymeric material may be substantially inelastic, intended to refer herein to the material having an elastic modulus of at least about 1 GPa. Optionally, the elastic modulus may be at least about 1.5 GPa, optionally at least about 2 GPa, optionally at least about 2.5 GPa. Optionally, the polymeric material has an elastic modulus of about 1 GPa to about 5 GPa, optionally about 1.5 GPa to about 4 GPa. Additionally or alternatively, the polymeric material may comprise a polymeric material having an ultimate tensile strength of at least about 70 MPa, optionally about 70 MPa to about 130 MPa, or about 80 MPa to about 120 MPa.

In some embodiments, the helically oriented depressions of the outer surface of the elongate tubular member may be filled with polymeric material such that at least about 50% of the depressions, in particular about 50 to about 100%, about 75% to about 100% or substantially all, of the helically oriented depressions, are at least partially filled with polymeric material.

In some embodiments, the polymeric material may form a layer on at least a part of the outer surface of the elongate tubular member. In some embodiments, the layer of polymeric material covers at least about 50% the outer surface of the elongate tubular member, in particular about 50 to about 100%, about 75% to about 100% or substantially all outer surface of the elongate tubular member. In some embodiments, the polymeric material may form a layer on at least a part of the outer surface of the elongate tubular member which is obtainable by heat shrinking a tube, a sleeve, or sheet of the polymeric material onto the outer surface of the elongate tubular member. In some embodiments, the polymeric material may form a layer on at least a part of the outer surface of the elongate tubular member which has a thickness of about 50 μm to about 400 μm, in particular about 75 μm to about 300 μm, or about 100 μm to about 200 μm.

In some embodiments, a polymeric material may form a layer on at least a part of the inner surface of the elongate tubular member. In some embodiments, the layer of polymeric material covers at least about 50% the inner surface of the elongate tubular member, in particular about 50 to about 100%, about 75% to about 100% or substantially all inner surface of the elongate tubular member. In some embodiments, the polymeric material may form a layer on at least a part of the inner surface of the elongate tubular member which is obtainable by radially expanding a heated tube of a polymeric material onto the inner surface of the elongate tubular member or by winding a plurality of metal strands onto polymeric material.

In some embodiments, the polymeric material may extend as a layer along at least a first portion of the length of the tubular member, and may be absent (or the layer removed) in a second portion of the length of the tubular member. The first portion may be longer than the second portion. By removing (or not providing) the polymeric material in selected regions, the flexibility may be increased in these regions because the polymer does not constrain freedom to flex. It is envisaged that the second region may correspond to one or more positions along the length of the elongate tubular member at which flexibility is desired to be increased locally.

In some embodiments, a portion of the plurality of metal strands may be joined to at least one circumferentially adjacent strand by welding, soldering, or brazing, or, alternatively, joined by a substantially inelastic adhesive. The metal strands may be joined to their respective neighboring strands such that sliding movements of adjacent strands are impaired and such that overall flexibility of the elongate tubular member is sufficiently retained to allow its use as a catheter tubing. Without wishing to be bound by theory, it is believed that sufficient flexibility of the elongate tubular member is retained if individual strands are joined to neighboring strands in only a few places. Such a joining pattern may stabilize the elongate tubular member such that its dimensional stability under compressive or extensive load may be improved. At the same time, sufficient localized sliding of strands is retained to allow bending of the elongate tubular member.

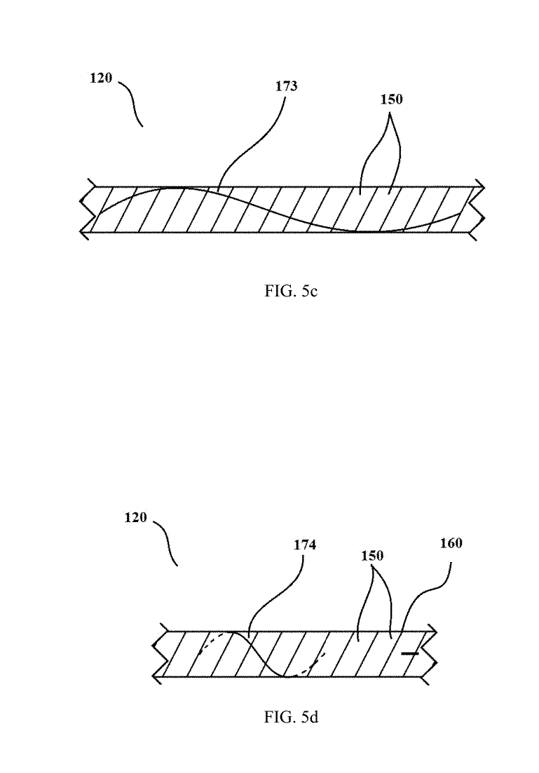

In some embodiments, at least a portion of the plurality of metal strands is welded at 1 to about 8 locations, in particular at 1 to about 4 locations, or about 2 to about 4 locations, to (a) circumferentially adjacent strand(s) per circumference of said strand. By way of example, the helical structure of an elongate tubular member may be laser-welded with a single linear longitudinally oriented welding line. Such a joining pattern is exemplarily shown in FIG. 5a. FIG. 5a schematically shows an elongate tubular member 120 having a plurality of helically wound strands 150 in a side-by-side arrangement defining an outer surface having a plurality of helically oriented depressions 160. The plurality of strands 150 is joined (e.g. laser-welded) to form a single linear longitudinally oriented joint pattern (e.g. welding line) 171. In this case, each single metal strand is laser-welded to its two neighbors once per circumference, i.e. a metal strand is welded at 2 locations to circumferentially adjacent strands per circumference of said strand. As a further example, two linear longitudinally oriented welding lines would result in 4 welding locations per circumference, and so on. As can be seen form the above, a location in the sense of the above disclosure is a point on the strand where the joint (e.g. the welding line) crosses over a depression 160 to contact a neighboring strand 150.

Suitable patterns which may retain sufficient flexibility are described in the following with reference to FIGS. 5a to 5d:

In some embodiments, the portion of the plurality of metal strands 150 may be joined to at least one circumferentially adjacent strand in a pattern of joints extending substantially continuously between the proximal end and the distal end of the elongate tubular member 120. An example of such a joint pattern is a continuous laser welding line 171 along or generally parallel to the axis of the elongate tubular member 120 as shown in FIG. 5a.

In some embodiments, the portion of the plurality of metal strands 150 may be joined to at least one circumferentially adjacent strand in a pattern of joints extending intermittently between the proximal end and the distal end of the elongate tubular member 120. An exemplary representation of such a joint pattern is the intermittent laser welding line 172 along or generally parallel to the axis of the elongate tubular member 120 as shown in FIG. 5b. In some embodiments (not shown in FIGS. 5a to 5d), the intermittent pattern of joints may be a series of annular rings, in particular at series of at least about 20 annular rings disposed between the proximal end and the distal end of the elongate tubular member.

In some embodiments, the portion of the plurality of metal strands 150 may be joined to at least one circumferentially adjacent strand in a pattern of joints which is longitudinally oriented in a straight or non-straight line 173 between the proximal end and the distal end of the elongate tubular member 120 as shown in FIG. 5c.

In some embodiments, the portion of the plurality of metal strands 150 may be joined to at least one circumferentially adjacent strand in a pattern of joints which is circumferentially oriented in a straight or non-straight line, a line which is helically oriented anti-clock-wise with respect to the helically wound plurality of metal strands 150, or a line which is helically oriented clock-wise with respect to the helically wound plurality of metal strands 150 but with a different pitch than the helically wound plurality of metal strands 150. The pattern of joints may extend between the proximal end and the distal end of the elongate tubular member 120. An exemplary embodiment of such a pattern of joints is shown in FIG. 5d. FIG. 5d shows a representation of an elongate tubular member 120 having a plurality of strands 150 defining an outer surface having a plurality of helically oriented depressions 160. In this case, the pattern of joints is a welding line 174 which is helically oriented anti-clock-wise with respect to the helically wound plurality of metal strands 150. The relative angle between the helically oriented depressions 160 and the line of helically oriented pattern of joints can be freely selected and may change along the elongate tubular member 120.

In some embodiments, the pattern of joints may be provided on at least part of the outer surface of the plurality of strands of an elongate tubular member. Alternatively or additionally, the pattern of joints may be provided on at least part of the inner surface of the elongate tubular member. In either case, it may extend between substantially the entire length of the elongate tubular member. The pattern of joints may be provided by any means known in the art, in particular by welding, soldering, or brazing, or, alternatively, by applying a substantially inelastic adhesive (e.g. duromers). The pattern of joints may be provided by welding, in particular by laser welding. In case that both a pattern of joints and a polymeric material is provided on the outer surface of the plurality of strands of an elongate tubular member, it will be understood that the pattern of joints may be applied first, i.e. the pattern of joints is at least partially covered by the polymeric material in surface areas where both a pattern of joints and polymeric material is disposed.

In some embodiments, a portion of the plurality of metal strands may be joined to at least one circumferentially adjacent strand on the outer surface of the elongate tubular member by a welding line, in particular by a laser welding line, wherein the welding line is helically oriented anti-clock-wise with respect to the helically wound plurality of metal strands or wherein the welding line is helically oriented clock-wise or a line which is helically oriented clock-wise with respect to the helically wound plurality of metal strands but with a different pitch than the helically wound plurality of metal strands. In some embodiments, a portion of the plurality of metal strands may be joined to at least one circumferentially adjacent strand on the outer surface of the elongate tubular member by a welding line, in particular by a laser welding line, wherein the welding line is helically oriented with a pitch that varies along the length of elongate tubular member. Varying the pitch along the length of elongate tubular member may provide regions of different flexibility, columnar strength and/or axial elongation. Exemplary pitches may include the range of about 0.5 to about 30 mm, in particular about 0.6 mm to about 20 mm, or about 0.7 mm to about 15 mm, or about 0.8 mm to about 10 mm.

In some embodiments, a combination of both pitch variation (or other welding pattern change) and absence of polymeric material layer, may be used to define zones of enhanced flexibility in local areas, for example to promote articulation at such positions.

By suitably applying one or more of the above described measures, undesirable elongation of the elongate tubular member under axial tensile loads may be reduced, and/or undesirable foreshortening under axial compression loads may be reduced. In some embodiments, the elongate tubular member may have an elongation along its longitudinal axis of less than about 0.5%, in particular about 0 to about 0.45%, or about 0.1 to about 0.4%, when subjected to a tensile load of about 100 N. Said tensile load may be applied along the longitudinal axis of the elongate tubular member.

In some embodiments, the delivery system may further comprise a load transmitting mount attached to the elongate tubular member at or approximate to its distal end, and/or a mount attached to the elongate tubular member at or approximate to its proximal end. The mount may be configured to transmit a tensile load of at least about 100 N to or from a further part of the delivery system which is attached or coupled to the mount. Examples of attached parts attached to the mount may include, for the distal end, housing components for the heart valve implant, a sheath, or another movable catheter part, and for the proximal end, a driver for interacting with an actuator of a handle for generating axial movement or other displacements.

In some embodiments, one or more mounts may be directly attached to the elongate tubular member. The polymeric layer may be absent (or removed) at the position of attachment. Load may be transmitted through or along the delivery system primarily by the elongate tubular member, and directly transmitted between the elongate tubular member and the mount.

In some embodiments, the delivery system may further comprise a second flexible elongate tubular member having a second proximal end and a second distal end and comprising a second interior lumen extending between the second proximal end and the second distal end, wherein the second elongate tubular member comprises a second plurality of metal strands which are arranged in a side-by-side relationship and helically wound along the longitudinal axis of the second elongate tubular member to define a second outer surface having a second plurality of helically oriented depressions. The second elongate tubular member may be arranged in the interior lumen of the first elongate tubular member. The first elongate tubular member may be arranged in the second interior lumen of the second elongate tubular member. In some embodiments, the second elongate tubular member may be further provided with features as described above for the first elongate tubular member.

In some embodiments, the delivery system may further comprise a housing (e.g. a sheath) for carrying the heart valve implant and a handle for operating the delivery system.

In some embodiments, the delivery system may be configured to at least partially re-collapse the heart valve implant after its at least partial expansion. In some embodiments, the delivery system may further comprise a sheath that is configured to at least partially re-collapse the heart valve implant by applying a tensile or compressive force to elongate tubular member.

In some embodiments, the delivery system may be configured to deliver a self-expandable or a balloon-expandable heart valve implant. In some embodiments, the delivery system may be configured to transfemorally deliver a heart valve implant. In some embodiments, the delivery system may be configured to transfemorally deliver a replacement valve for the aortic heart valve.

Although described with respect to a delivery system for delivering an expandable heart valve implant, it will be appreciated that the elongate tubular member described herein can also be provided on other medical devices and in particular other catheters in which high compressive and/or tensile may have to be applied or transmitted.

One aspect of the disclosure describes a method of repositioning or withdrawing an at least partially expanded implant during the delivery of an expandable heart valve implant to a target site in a patient comprising applying a tensile force or compressive to the elongate tubular member of a delivery system as described above which causes the at least partially expanded heart valve implant to re-collapse at least partially, followed by repositioning or withdrawing of the implant.

In some embodiments, the method of repositioning or withdrawing an at least partially expanded implant may further comprise the use of a delivery system which is provided with features as described above for delivery system and/or the elongate tubular member.

One aspect of the disclosure describes a method for manufacturing a delivery system as described above, comprising providing a flexible elongate tubular member having a proximal end and a distal end and comprising an interior lumen extending between the proximal end and the distal end, wherein the elongate tubular member comprises a plurality of metal strands which are arranged in a side-by-side relationship and helically wound along the longitudinal axis of the elongate tubular member to define an outer surface having a plurality of helically oriented depressions; and: i) providing a polymeric material, and, in any order: heating the polymeric material to a flowable state, and applying the polymeric material onto at least a part of the outer surface of the elongate tubular member, such that at least part of the helically oriented depressions of the outer surface are at least partially filled; or ii) joining a portion of the plurality of metal strands to at least one circumferentially adjacent strand by welding, soldering, or brazing.

It should be understood that although the above discussion was focused on a delivery system for a heart valve implant, i.e. for use within the vascular system and/or the heart of a patient, other embodiments of medical devices or methods in accordance with the disclosure can be adapted and configured for use in other parts of the anatomy of a patient. For example, devices and methods in accordance with the disclosure can be adapted for use in the digestive or gastrointestinal tract, such as in the mouth, throat, small and large intestine, colon, rectum, and the like. For another example, devices and methods can be adapted and configured for use within the respiratory tract, such as in the mouth, nose, throat, bronchial passages, nasal passages, lungs, and the like. Similarly, the devices and/or medical devices described herein with respect to percutaneous deployment may be used in other types of surgical procedures as appropriate. Devices and methods in accordance with the invention can also be adapted and configured for other uses within the anatomy.

Another aspect of the disclosure pertains to a delivery system for transcatheter delivery of a heart implant capable of tracking along a significantly curved path, for example, a system for transfemoral delivery of a heart valve implant which is capable of reliably tracking around the aortic arch, or transeptal delivery capable of reliably tracking a curved path near the septum.

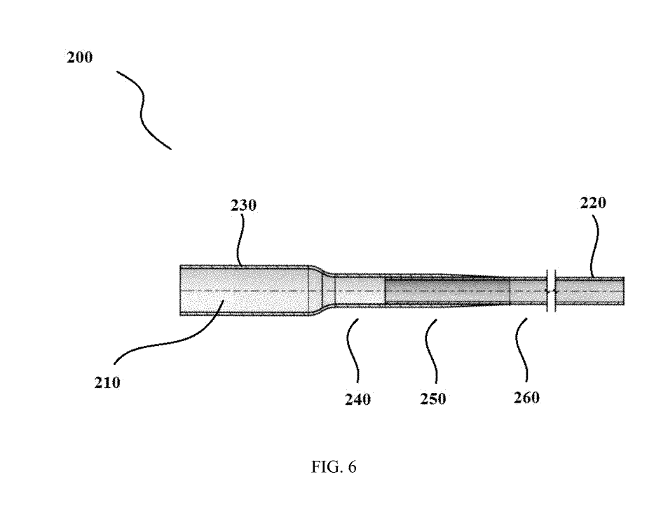

As seen in FIG. 6, in one embodiment, a catheter 200 for transcatheter (e.g. transfemoral) delivery of a heart valve implant 210 (not shown in detail) may comprise an elongate tubular member 220 having a proximal end and a distal end; a housing component 230 for carrying the heart valve implant 210 which is attached to the distal end of the elongate tubular member 220. The housing component 230 may comprise a first section 240 at or approximate to the distal end of the elongate tubular member 220, and a second section 250 proximally adjacent to the first section 240. The first section 240 may have a length in longitudinal direction of between about 5 and about 50 mm. The first section may have an internal diameter of between about 2 and about 6 mm. The first section may be more flexible than the second section 250. The second section may be configured to bend, in particular without kinking, to an angle of at least about 45° relative to its longitudinal axis.

In the context of the present disclosure, bending without kinking may refer to a bending behavior of the first section wherein the curvature caused by bending the first section at its proximal and distal end is continuous and without abrupt changes. Alternatively or additionally, bending without kinking may refer to a bending behavior of the first section wherein bending does not substantially change the inner diameter of the first section and/or does not substantially change the cross-sectional area of the inner lumen of the first section. Alternatively or additionally, bending without kinking may refer to a bending behavior of the first section wherein a metal ring having an internal diameter of between about 101% and about 110%, and in particular about 105%, of the outer diameter of the first section 240 in the non-bent state can be passed over the entirety of the first section in the bent state. Alternatively or additionally, bending without kinking may refer to a bending behavior of the first section wherein a wire having an outer diameter of between about 90% and about 99%, and in particular about 95%, of the inner diameter of the first section in the non-bent state can be moved throughout the entirety of the first section in the bent state.

In the context of the present disclosure, greater flexibility of the first section in comparison to the second section may refer to a comparison of the resistance of the first section to bending of the first section to an angle of at least about 45° relative to its longitudinal axis to the resistance of the second section to bending of the second section to an angle of at least about 45° relative to its longitudinal axis. The resistance may, for instance, be determined by measuring the force (in e.g. Newton) required for bending a section at its proximal and distal end to e.g. an angle of e.g. about 45° relative to its longitudinal axis.

In the context of the present disclosure, the housing component and the elongate tubular member are located in proximity to each other and may overlap at their proximal and distal end, respectively. Thus, although the present disclosure assigns the first and second sections and to be part of the housing component, these sections may perform functionalities or be adapted to be part of the elongate tubular member.

Some embodiments may combine the elongate tubular member or the housing component described above with a helically wound metallic strand elongate tubular member described hereinbefore. The helically wound metallic strand elongate tubular member may be disposed within or passing through the housing component. The helically wound metallic strand elongate tubular member may optionally be a second elongate tubular member different from the elongate tubular member attached to the housing component. In at least one operative position of the housing component (the housing component and the helically wound metallic strand tubular member may optionally be displaceable in the axial direction, one relative to the other), the first section may be generally aligned or axially in register with a region of the (e.g. second) elongate tubular member that also is configured with enhanced flexibility with respect to a further region of the (e.g. second) elongate tubular member. As explained earlier, enhanced flexibility of a region of the (e.g. second) elongate tubular member may be provided by pitch or pattern variation of a joining pattern of adjacent strands, and/or by absence in local regions of a polymeric constraining sleeve. Such a configuration of both the housing component and an (e.g. second) elongate tubular member may provide a delivery system with a localized region of enhanced flexibility near its distal end despite the delivery system comprising multiple components and members nested one within another.

As also exemplarily shown in FIG. 6, additionally or alternatively to any of the above, in one embodiment, the catheter for transfemoral delivery 200 of a heart valve implant 210 may comprise an elongate tubular member 220 having a proximal end and a distal end and a housing component 230 for carrying the heart valve implant which is attached to the distal end of the elongate tubular member 220. The housing component 230 may comprise a first section 240 at or approximate to its distal end and a second section 250 proximally adjacent to the first section 240. The first section 240 may have a length in longitudinal direction of between about 5 and about 50 mm, an internal diameter of between about 2 and about 6 mm. The first section 240 may have a reduced wall thickness in comparison to the second section 250.

In some embodiments, the first section may have a reduced wall thickness in comparison to the second section. In this context, wall thickness may refer to the minimum wall thickness of the first section and second section, respectively. It may also refer to the maximum thickness or an average thickness over the length of the respective section. The wall thickness of the first section may be in the range of about 25% to about 95%, in particular from about 30% to about 85%, or from about 35% to about 75%, or about 40% to about 65%, in relation to the wall thickness of the second section. Without wishing to be bound by theory, it will be appreciated that a reduced wall thickness may increase the flexibility of an elongate tubular member.

As also exemplarily shown in FIG. 6, in some embodiments, the housing component 230 member may comprise a third section 260 proximally adjacent to the second section 250, wherein the first section 240 comprises a first material and the third section 260 comprises a second material which is different from the first material.

As also exemplarily shown in FIG. 6, in some embodiments, the second section 250 may comprise or consist of a region in which a tubular section comprising the first material and a tubular section comprising the second material overlap.

In some embodiments, the third section may be more flexible than the second section. In some embodiments, the first section may be more flexible than the second section and the third section may be less flexible than the first section. In this context, greater or lesser flexibility may refer to flexibility as described above.

In some embodiments, the second section may have a length in longitudinal direction of between about 2 and about 50 mm and an internal diameter of between about 2 and about 6 mm.

In some embodiments, the housing component may comprise a sheath which comprises the same material as the first section.

In some embodiments, the third section comprises a reinforcement, in particular a braid-, coil- or ring-reinforcement.

In some embodiments, the first section and/or second section may comprise a polyether-block-amid-copolymer or a low-density polyethylene (LDPE) and the third section may comprise a polyamide or a high-density polyethylene (HDPE).

In some embodiments, the first section may have a length in longitudinal direction of between about 5 and about 20 mm. The first section may be configured bend without kinking to an angle of at least about 90° relative to its longitudinal axis.

In some embodiments, a means for actively bending the first section which is operable at a catheter handle may be absent from the catheter. Such a catheter may be referred to as non-steerable catheter. Instead, the catheter may be configured to be guided substantially only by a guidewire along which the catheter tracks to the implantation site.

One aspect of the disclosure describes a catheter system comprising the catheter as described above, a handle, and optionally an implantable heart valve.

In some embodiments, the catheter system may be provided with features as described above for the catheter for transfemoral delivery.

One aspect of the disclosure describes a method of manufacturing a catheter or catheter system as described above, wherein a first and a second tubular member may be provided and wherein the first and the second tubular member may be attached to each other.

In some embodiments, the method may further be implemented to provide a catheter for transfemoral delivery which is provided with features as described features as described above.

It should be understood that although the above discussion was focused on a catheter for transfemoral delivery of a heart valve implant, i.e. for use within the vascular system and/or the heart of a patient, other embodiments of medical devices or methods in accordance with the disclosure can be adapted and configured for use in other parts of the anatomy of a patient. For example, devices and methods in accordance with the disclosure can be adapted for use in the digestive or gastrointestinal tract, such as in the mouth, throat, small and large intestine, colon, rectum, and the like. For another example, devices and methods can be adapted and configured for use within the respiratory tract, such as in the mouth, nose, throat, bronchial passages, nasal passages, lungs, and the like. Similarly, the devices and/or medical devices described herein with respect to percutaneous deployment may be used in other types of surgical procedures as appropriate. Devices and methods in accordance with the invention can also be adapted and configured for other uses within the anatomy.

The following list of clauses pertains to a delivery system for transfemoral delivery of a heart valve implant which is capable of safely tracking around the aortic arch.

1. A catheter for transfemoral delivery of a heart valve implant comprising:
an elongate tubular member having a proximal end and a distal end;
a housing component for carrying the heart valve implant which is attached to the distal end of the elongate tubular member;
wherein the housing component comprises a first section at or approximate to the distal end of the elongate tubular member, and a second section proximally adjacent to the first section;
wherein the first section has a length in longitudinal direction of between about 5 and about 50 mm, an internal diameter of between about 2 and about 6 mm, is more flexible than the second section, and is configured to bend without kinking to an angle of at least about 45° relative to its longitudinal axis.

2. A catheter for transfemoral delivery of a heart valve implant comprising:
an elongate tubular member having a proximal end and a distal end;
a housing component for carrying the heart valve implant which is attached to the distal end of the elongate tubular member;
wherein the housing component comprises a first section at or approximate to the distal end of the elongate tubular member, and a second section distally adjacent to the first section;
wherein the first section has a length in longitudinal direction of between about 5 and about 50 mm, an internal diameter of between about 2 and about 6 mm, and a reduced wall thickness in comparison to the second section.

3. The catheter according to clause 1, wherein the first section has a reduced wall thickness in comparison to the second section.

4. The catheter according to clause 1 or 3, wherein the housing component comprises a third section proximally adjacent to the second section, wherein the first section comprises a first material and the third section comprises a second material which is different from the first material.

5. The catheter according to clause 4, wherein the second section comprises or consists of a region in which a tubular section comprising the first material and a tubular section comprising the second material overlap.

6. The catheter according to clause 4 or 5, wherein the third section is more flexible than the second section.

7. The catheter according to any of the clauses 3 to 5, wherein the first section is more flexible than the second section and the third section is less flexible than the first section.

8. The catheter according to any of the clauses 1 to 7, wherein the second section has a length in longitudinal direction of between about 2 and about 50 mm and an internal diameter of between about 2 and about 6 mm.

9. The catheter according to any of the clauses 1 to 8, wherein the housing component comprises a sheath which comprises the same material as the first section.

10. The catheter according to any of the clauses 1 to 9, wherein the third section comprises a reinforcement, in particular a braid-, coil- or ring-reinforcement.

11. The catheter according to any of the clauses 1 to 10, wherein the first section comprises a polyether-block-amid-copolymer or a LDPE and the third section comprises a polyamide or a HDPE.

12. The catheter according to any of the clauses 1 to 11, wherein the first section has a length in longitudinal direction of between about 5 and about 20 mm and is configured bend without kinking to an angle of at least about 90° relative to its longitudinal axis.

13. The catheter according to any of the clauses 1 to 12, wherein the catheter does not comprise a means for actively bending the first section which is operable at a catheter handle.

14. A catheter system comprising the catheter according to any of the clauses 1 to 12, a handle, and optionally an implantable heart valve.

15. A method of manufacturing a catheter or catheter system according to any of the clauses 1 to 14, wherein a first and a second tubular member are provided and wherein the first and the second tubular member are attached to each other.

Another aspect of the disclosure relates to a delivery catheter comprising an interface member. During valve deployment, some devices may open a protective sheath in distal direction potentially creating annular gaps between the replacement valve and the mounting components during deployment. An interface member may be used to avoid such annular gaps. Exemplary interface members are described in WO 2014/122205 A1 and WO 2012/038550 A1. Both disclosures are incorporated herein by reference. An interface member may be radially collapsible when covered by the distal sheath and self-expands when the distal sheath is opened clear of the valve mounting component. When expanded, the interface member may provide a smooth interface surface between the mounting component and the distal sheath, avoiding annular gaps after deployment. Such an interface member may elongate axially when it is compressed by the distal sheath when closed. This means that additional space may have to be provided in the valve housing of the delivery device, distally of the stent holder to accommodate the elongation. Additional space means that the distal part of the delivery device may be longer than necessary and may enter deeper in sensitive organ parts (such as the ventricle) than necessary.

This aspect of the disclosure pertains to a delivery catheter comprising a holder for engaging the valve in the collapsed state of the valve, a sheath translatable with respect to the holder between a first position in which the sheath surrounds at least a first portion of the holder, and a second position in which the sheath does not surround the first portion of the holder, the delivery catheter further comprising an interface member.

The interface member may be biased towards a radially expanded condition for (i) bridging a gap between the holder and the sheath when in the second position, and/or (ii) defining a generally smooth interface surface between the holder and the sheath when in the second position.

The interface member may comprise any one or more of the following, which are all optional:
the interface member may have an axially lengthened condition and an axially shortened condition, the axially lengthen condition corresponding to the expanded condition, and the axially shortened condition corresponding to a radially contracted condition when the sheath is in the first position.
the interface member may comprise a plurality of cantilever elements biased to the radially expanded condition and collapsible to a collapsed condition when the sheath is in the first position.
in the collapsed condition, the cantilever elements have an axially shorter profile than in the expanded condition.
the tips of the cantilever elements are generally positioned radially inward relative to a fixed and/or less mobile end of the cantilever elements (e.g. in the radially expanded condition and/or radially collapsed condition).

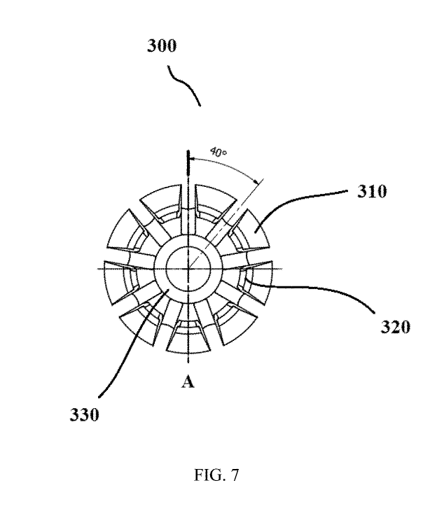

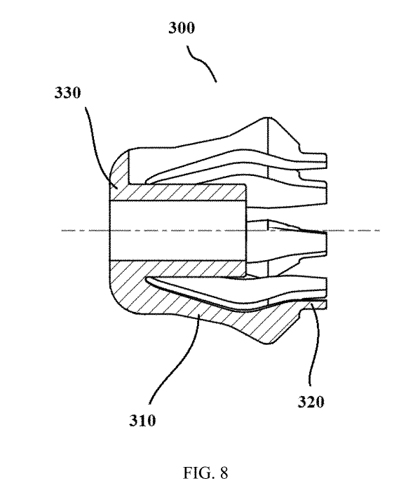

An exemplary embodiment of this aspect of the disclosure is shown in FIGS. 7 and 8.

FIG. 7 shows a front view of an exemplary interface member 300. The interface member 300 has a plurality of cantilever elements 310, in this specific example nine cantilever elements which are arranged around a central hub 330. The nine cantilever elements are circumferentially arranged at angles of 40°, 80°, 120°, 160°, 200°, 240°, 280°, 320°, and 360°, respectively. Line A indicates the cross-sectional plane shown in FIG. 8.

FIG. 8 is a cross-sectional view of the interface member 300 as shown in FIG. 7. The cross-section is taken along plane A shown in FIG. 7. Interface member 300 has a plurality of cantilever elements 310 which terminate in tips 320. The cantilever elements 310 may be joined at or form a central hub 330 at the proximal end of the interface member 300. At the distal end of the interface member 300, the tips 320 of the plurality of cantilever elements 310 may form crown-like structure. FIG. 8 shows the interface member 300 in the expanded configuration. The plurality of flexible cantilever elements 310 may be compressed by e.g. crimping. The plurality of flexible cantilever elements 310 may be flexible and/or be shaped such that the tips 320 perform a circular motion towards the central longitudinal axis of the interface member 300. Crimping the interface member 300 may shorten or keep constant the distance between the proximal end and the distal end of the interface member 300. When interface member 300 reverts to expanded configuration, the plurality of cantilever elements 310 again expand radially outward and the plurality of tips 320 again perform a circular motion.

The following list of clauses pertains to an interface member. Although referring to a specific interface member in clause 1, the features indicated in the below clauses 2 to 15 are freely combinable with any of the other above-disclosed embodiments.

1. An interface member for a system for delivering a medical device to a target site in a patient, the interface member having a distal end and a proximal end and comprising:
a hub at the proximal end, and
a plurality of flexible cantilever elements which are attached to the hub, extend towards the distal end of the interface member and terminate in tips; and
wherein the interface member has a collapsed and an expanded configuration, wherein the plurality of flexible cantilever elements expand radially outward and the plurality of tips perform a circular motion when the interface member assumes the expanded configuration

2. The interface member according to clause 1, wherein the interface member comprises at least about 3 cantilever elements, in particular between about 3 and about 20 cantilever elements.

3. The interface member according to clause 1 or 2, wherein the plurality of cantilever elements and optionally the hub form a unitary body.

4. The interface member according to any of the clauses 1 to 3, wherein the hub has a substantially circular shape and/or wherein the hub is tapering towards the proximal end.

5. The interface member according to any of the clauses 1 to 4, wherein the shape and/or flexibility of the cantilever elements is adapted such that the cantilever elements are more flexible towards the hub.

6. The interface member according to any of the clauses 1 to 5, wherein the cross-sectional dimensions of the cantilever elements are adapted such that the cantilever elements are more flexible towards the hub.

7. The interface member according to any of the clauses 1 to 6, wherein the shape and/or flexibility of the cantilever elements is adapted such that the cantilever elements bend at or approximate to the hub when the interface member assumes the collapsed configuration.

8. The interface member according to any of the clauses 1 to 7, wherein shape and/or flexibility of the cantilever elements is adapted such that the cantilever elements pivot about a point which is radially distant from the central axis of the interface member.

9. The interface member according to any of the clauses 1 to 8, wherein the interface member shortens in longitudinal direction when assuming the collapsed configuration.

10. The interface member according to any of the clauses 1 to 9, wherein the tips form a crown.

11. The interface member according to any of the clauses 1 to 10, wherein in the expanded configuration the tips are positioned at a radial distance to the central axis of between about 2 and about 10 mm.

12. The interface member according to any of the clauses 1 to 11, wherein the interface member has a symmetrical bulbus-like or an asymmetrical bulbus-like shape in the expanded configuration.

13. The interface member according to any of the clauses 1 to 12, wherein the interface member is a unitary body composed of polymeric material.

14. The interface member according to any of the clauses 1 to 13, wherein the interface member is self-expandable from the collapsed to the expanded configuration.

15. A system for delivering a medical device to a target site in a patient, the system comprising a retractable housing which accommodates an implant having a collapsed and an expanded configuration and an interface member according to any of the clauses 1 to 14.

Those skilled in the art will recognize that aspects of the present disclosure may be manifested in a variety of forms other than the specific embodiments described and contemplated herein. Accordingly, departure in form and detail may be made without departing from the scope and spirit of the present disclosure as described in the appended claims. This may include, to the extent that it is appropriate, the use of any of the features of one example embodiment or aspect being used in other embodiments or aspects.

1. A delivery system for delivering an expandable heart valve implant to a target site in a patient comprising: a flexible elongate tubular member having a proximal end and a distal end, an interior lumen extending between the proximal end and the distal end, and a polymeric material;
wherein the elongate tubular member comprises a plurality of metal strands which are arranged in a side-by-side relationship and helically wound along the longitudinal axis of the elongate tubular member to define an outer surface having a plurality of helically oriented depressions; and
wherein the polymeric material is disposed on the elongate tubular member such that at least part of the helically oriented depressions of the outer surface of the elongate tubular member are at least partially filled.

2. The delivery system of claim 1, wherein the polymeric material forms a layer on at least a part of the outer surface of the elongate tubular member.

3. The delivery system of claim 1, wherein at least a portion of the plurality of metal strands has a non-circular cross-sectional shape, in particular a rectangular shape, a rectangular shape with rounded edges, an oval shape, a trapezoidal shape, or a trapezoidal shape with rounded edges or rounded corners.

4. The delivery system of claim 1, wherein the elongate tubular member comprises a single layer of helically wound metal strands or wherein the elongate tubular member comprises two layers of helically wound metal strands.

5. The delivery system of claim 1, wherein a portion of the plurality of metal strands are joined to at least one circumferentially adjacent strand by welding, soldering, brazing, or by a substantially inelastic adhesive.

6. The delivery system of claim 5, wherein the portion of the plurality of metal strands are joined to at least one circumferentially adjacent strand in a pattern of joints and wherein said pattern is:
extending substantially continuously between the proximal end and the distal end of the elongate tubular member; and/or
extending intermittently between the proximal end and the distal end of the elongate tubular member; and/or
longitudinally oriented in a straight or non-straight line between the proximal end and the distal end of the elongate tubular member; and/or
circumferentially oriented in a straight or non-straight line between the proximal end and the distal end of the elongate tubular member; and/or
a line which is helically oriented anti-clock-wise with respect to the helically wound plurality of metal strands; and/or
line which is helically oriented clock-wise with respect to the helically wound plurality of metal strands but with a different pitch than the helically wound plurality of metal strands; and/or
such that at least a portion of the plurality of metal strands is welded at 1 to about 8 locations to a circumferentially adjacent strand per circumference of said strand;
or a combination thereof.

7. The delivery system of claim 1, wherein the elongate tubular structure has an elongation along its longitudinal axis of less than about 0.5% when subjected to a tensile load of about 100 N; or wherein the delivery system further comprises a mount attached to the elongate tubular member at or approximate to its distal end which is configured to transmit a tensile load of at least about 100 N to a further part of the delivery system which is attached to the mount.

8. The delivery system of claim 1, further comprising a second flexible elongate tubular member having a second proximal end and a second distal end and comprising a second interior lumen extending between the second proximal end and the second distal end, wherein the second elongate tubular member comprises a second plurality of metal strands which are arranged in a side-by-side relationship and helically wound along the longitudinal axis of the second elongate tubular member to define a second outer surface having a second plurality of helically oriented depressions; wherein the second elongate tubular member is arranged in the interior lumen of the first elongate tubular member or wherein the first elongate tubular member is arranged in the second interior lumen of the second elongate tubular member.

9. Method of repositioning or withdrawing an at least partially expanded implant during the delivery of an expandable heart valve implant to a target site in a patient comprising applying a tensile force to the elongate tubular member of a delivery system according to claim 1 which causes the at least partially expanded heart valve implant to re-collapse at least partially, followed by repositioning or withdrawing of the implant.

10. Method for manufacturing a delivery system according to claim 1, comprising:
providing a flexible elongate tubular member having a proximal end and a distal end and comprising an interior lumen extending between the proximal end and the distal end, wherein the elongate tubular member comprises a plurality of metal strands which are arranged in a side-by-side relationship and helically wound along the longitudinal axis of the elongate tubular member to define an outer surface having a plurality of helically oriented depressions;
and providing a polymeric material, and, in any order: heating the polymeric material to a flowable state, and applying the polymeric material onto at least a part of the outer surface of the elongate tubular member, such that at least part of the helically oriented depressions of the outer surface are at least partially filled.

11. A delivery system for delivering an expandable heart valve implant to a target site in a patient comprising: a flexible elongate tubular member having a proximal end and a distal end, and an interior lumen extending between the proximal end and the distal end;
wherein the elongate tubular member comprises a plurality of metal strands which are arranged in a side-by-side relationship and helically wound along the longitudinal axis of the elongate tubular member to define an outer surface having a plurality of helically oriented depressions; and wherein a portion of the plurality of metal strands are joined to at least one circumferentially adjacent strand by welding, soldering, or brazing.

12. The delivery system of claim 11, wherein the flexible elongate tubular member comprises a polymeric material which is disposed on the elongate tubular member such that at least part of the helically oriented depressions of the outer surface of the elongate tubular member are at least partially filled.

13. The delivery system of claim 11, wherein at least a portion of the plurality of metal strands has a non-circular cross-sectional shape, in particular a rectangular shape, a rectangular shape with rounded edges, an oval shape, a trapezoidal shape, or a trapezoidal shape with rounded edges or rounded corners.

14. The delivery system of claim 11, wherein the elongate tubular member comprises a single layer of helically wound metal strands or wherein the elongate tubular member comprises two layers of helically wound metal strands.

15. The delivery system of claim 11, wherein the portion of the plurality of metal strands are joined to at least one circumferentially adjacent strand in a pattern of joints and wherein said pattern is:
extending substantially continuously between the proximal end and the distal end of the elongate tubular member; and/or
extending intermittently between the proximal end and the distal end of the elongate tubular member; and/or
longitudinally oriented in a straight or non-straight line between the proximal end and the distal end of the elongate tubular member; and/or
circumferentially oriented in a straight or non-straight line between the proximal end and the distal end of the elongate tubular member; and/or
a line which is helically oriented anti-clock-wise with respect to the helically wound plurality of metal strands; and/or
line which is helically oriented clock-wise with respect to the helically wound plurality of metal strands but with a different pitch than the helically wound plurality of metal strands; and/or
such that at least a portion of the plurality of metal strands is welded at 1 to about 8 locations to a circumferentially adjacent strand per circumference of said strand;
or a combination thereof.

16. The delivery system of claim 11, wherein the elongate tubular structure has an elongation along its longitudinal axis of less than about 0.5% when subjected to a tensile load of about 100 N; or wherein the delivery system further comprises a mount attached to the elongate tubular member at or approximate to its distal end which is configured to transmit a tensile load of at least about 100 N to a further part of the delivery system which is attached to the mount.

17. The delivery system of claim 11, further comprising a second flexible elongate tubular member having a second proximal end and a second distal end and comprising a second interior lumen extending between the second proximal end and the second distal end, wherein the second elongate tubular member comprises a second plurality of metal strands which are arranged in a side-by-side relationship and helically wound along the longitudinal axis of the second elongate tubular member to define a second outer surface having a second plurality of helically oriented depressions; wherein the second elongate tubular member is arranged in the interior lumen of the first elongate tubular member or wherein the first elongate tubular member is arranged in the second interior lumen of the second elongate tubular member.

18. The delivery system of claim 11, wherein the delivery system is configured to at least partially re-collapse the heart valve implant after its at least partial expansion.

19. Method of repositioning or withdrawing an at least partially expanded implant during the delivery of an expandable heart valve implant to a target site in a patient comprising applying a tensile force to the elongate tubular member of a delivery system according to claim 11 which causes the at least partially expanded heart valve implant to re-collapse at least partially, followed by repositioning or withdrawing of the implant.

20. Method for manufacturing a delivery system according to claim 11, comprising:
providing a flexible elongate tubular member having a proximal end and a distal end and comprising an interior lumen extending between the proximal end and the distal end, wherein the elongate tubular member comprises a plurality of metal strands which are arranged in a side-by-side relationship and helically wound along the longitudinal axis of the elongate tubular member to define an outer surface having a plurality of helically oriented depressions;
and joining a portion of the plurality of metal strands to at least one circumferentially adjacent strand by welding, soldering, or brazing.
